(19) United States
(12) Patent Application Publication
(10) Pub. No.: US 2020/0411146 A1
(43) Pub. Date: Dec. 31, 2020

(54) EHR DATABASE INDEXING AND DATA RETRIEVAL

(71) Applicant: MIRR LLC, Houston, TX (US)

(72) Inventors: David McEwing, Houston, TX (US); Omar K. Higgs, Los Angeles, CA (US)

(73) Assignee: MIRR LLC, Houston, TX (US)

(21) Appl. No.: 17/015,267

(22) Filed: Sep. 9, 2020

Related U.S. Application Data
(63) Continuation-in-part of application No. 16/859,260, filed on Apr. 27, 2020.
Application No. 16/859,260 is a continuation-in-part of application No. 16/532,152, filed on Aug. 5, 2019.
(60) Provisional application No. 62/923,676, filed on Oct. 21, 2019.
(60) Provisional application No. 62/942,827, filed on Dec. 3, 2019.
(60) Provisional application No. 62/942,841, filed on Dec. 3, 2019.
(60) Provisional application No. 62/714,845, filed on Aug. 6, 2018.
(60) Provisional application No. 62/728,756, filed on Sep. 8, 2018.
(60) Provisional application No. 62/750,300, filed on Oct. 25, 2018.

(51) Int. Cl. G16H 10/60; G06F 16/9538; G06N 20/00

(52) U.S. Cl. CPC G16H 10/60; G06N 20/00; G06F 16/9538

(57) ABSTRACT

Database of patient specific electronic health records indexed by ICD or CPT medical coding protocols or similar. The indexing allows organization of the patient's records within the database by health diagnoses, treatments or tests. This method facilitates rapid searching and retrieval of past diagnoses, etc. for utilization in diagnosis, treatment or testing of the patient in subsequent medical events. The method allows a clinician or health care provider to rapidly learn whether a patient has a history of prior treatment for the symptoms now experienced. The software and method can be utilized to search billing records wherein the ICD or similar codes have been recorded and then match the coded events to events contained in the patient's EHR database. This allows searching of the EHR database utilizing diagnosis, treatment and test codes in situations where old patient records stored in the database have not adopted the code indexing.

### RELATED APPLICATIONS

This application is a CONTINUATION IN PART of pending application Ser. No. 16/859,260 filed Apr. 27, 2020 and entitled Diagnostic And Treatment Tool And Method For Electronic Recording And Indexing Patient Encounters For Allowing Instant Search Of Patient History and which is incorporated by reference herein in its entirety.

This application is also a CONTINUATION IN PART of pending application Ser. No. 16/532,152 filed Aug. 5, 2019 entitled “INDEXING OF MEDICAL RECORDS UTILIZING CODING PROTOCOLS”. This application claims priority to the Ser. No. 16/532,152 application which is incorporated by reference herein in its entirety.

The Ser. No. 16/532,152 application claims priority to provisional application Ser. No. 62/714,845, entitled “INDEXING OF MEDICAL RECORDS UTILIZING CODING PROTOCOLS” and filed Aug. 6, 2018, said application is incorporated herein by reference in its entirety. The Ser. No. 16/532,152 application also claims priority to provisional application Ser. No. 62/728,756, entitled “INDEXING OF MEDICAL RECORDS UTILIZING CODING PROTOCOLS”, filed Sep. 8, 2018 and said application is also incorporated herein by reference in its entirety. Further, the Ser. No. 16/532,152 application claims priority to provisional application Ser. No. 62/750,300 entitled “INDEXING OF MEDICAL RECORDS UTILIZING CODING PROTOCOLS” filed Oct. 25, 2018, said application being incorporated herein by reference in its entirety.

This application also claims priority to provisional applications 62/923,676 filed Oct. 21, 2019, entitled “Disclosure of Reimbursement with Suggested Code Display”, 62/942,827 filed Dec. 3, 2019, and entitled “Display or Search of Treatment and Test Codes Correlated to Treatment”, and 62/942,841 filed Dec. 3, 2019 entitled “Display or Search of Diagnosis, Treatment and Test Codes”. These three provisional applications are incorporated herein by reference in their entirety.

### FIELD OF USE

This disclosure pertains solving the problem of obtaining a patient medical history of diagnosis, treatment and testing in real-time during a patient encounter. Timely receipt of the relevant portions of a patient's medical history greatly assists in creating the correct diagnosis of the patient's present condition or malady observed in the current patient encounter. Creation of the correct diagnosis facilitates timely treatment of the patient's medical condition or malady.

This disclosure pertains to the indexing patient medical information or medical history using an established and authoritative medical code protocol.

The disclosure also pertains to submission of search requests into the patient's history utilizing this indexing for real-time data retrieval from databases or repositories of patient medical records (hereinafter EHR Database). The unique indexed database will allow immediate access to relevant portions of electronically stored patient records of past medical treatment or history in situations of medical emergency or other critical times. The records will be immediately accessible to a treating clinician. The access to records will be made without human intervention. This disclosure allows treatment of the cause and not merely the symptoms of a patient's malady.

This disclosure achieves creation of a new indexed database of patient medical records. This improved new database will allow immediate electronic searching of a patient's medical history relevant to a currently observed medical condition. It will be appreciated that, increasingly, patient medical records are collected and stored by digital or electronic means, i.e., EHRs. This results in the creation of electronic databases that are electronically readable and searchable. The records may be created and stored by individual physicians, multi-physician staffed clinics or medical groups, hospital and medical centers.

There is no central repository of patient medical data or uniform methodology for creating databases for patient medical information. Accordingly, a patient may have records of his/her medical treatment stored at multiple facilities and geographic locations. The medical history of maladies may include records of past treatment for illnesses, surgeries, e.g., appendectomy, or medical conditions, e.g., high blood pressure or heart conditions.

This disclosure teaches a method of creating an electronically searchable index for these historical records which will allow a present day clinician receiving a patient for examination or treatment to instantly access relevant portions of a patient's history stored in multiple databases.

It will be appreciated that without access to a relevant portions of a patient's medical history, the present day clinician may only treat the observed symptoms in contrast to the underlying problem or cause.

### RELATED PRIOR ART

A medical health care provider creates a record of patient diagnosis and treatment. The record is individualized by patient. The medical records are patient specific. These records may be hand written and unindexed (other than perhaps by patient name or chronological order).

It is difficult for a subsequent medical health care provider (hereinafter “clinician”) to rapidly obtain access to the patient's records of earlier test, treatments or diagnosis that were conducted by separate health care providers. Requests may require the patient's written consent and this consent must be provided to the earlier health care provider before there can be any release of patient records.

Health information exchanges (HIES) have also been created among certain medical health care providers to facilitate sharing of patient medical records. See for example Greater Houston Health Connect, http://www.ghhconnect.org.

There has been recent increasing effort to record and transform all patient records into an electronic readable format. Reference is made to the Affordable Care Act, Public Law 111-148, which contained financial incentives for transforming and creation of systems for sharing electronic patient records. Various efforts have been made to create mechanisms for automatically and electronically transcribing orally dictated clinician notes for storage and evaluation by electronic means. See for example the Yegnanarayanan U.S. Pat. No. 9,904,768 issued Feb. 27, 2018. See also the published application of Snider 2018/0081859 published Mar. 22, 2018.

Alphanumeric medical coding protocols have been in existence for multiple years and are trusted and relied upon by health care providers and third party payors for reimbursement for health care services. Protocols include the International Classification of Diseases (ICD), the Current Procedural Terminology (CPT) and the Healthcare Common Procedure Coding System (HCPCS) code protocol. These protocols comprise alphanumeric codes with text descriptions and providing a comprehensive listing of possible medical diagnoses, treatments and tests. The text descriptions associated with each alphanumeric code are variously referenced as “code descriptors”. It will be appreciated that the code protocols have been utilized by the insurance industry to ensure identical but differently clinician described treatments are “normalized” for uniformity of payment.

### BRIEF SUMMARY OF DISCLOSURE

Large and disparate electronic databases exist containing individualized patient records of past medical health care services. These records comprise a voluminous history of all medical diagnoses, treatments or tests of a patient over the patient's lifetime.

These electronically recorded and stored patient medical records are termed electronic medical records (EMR) or electronic health records (EHR). The terms are sometimes used synonymously. However EMR typically refers to a patient's medical records stored at a single medical facility, i.e., a clinic or individual physician's office. An EHR refers to the collective electronic medical records stored at within multiple databases. This disclosure will utilize “EHR database” to refer to both EMR and EHR record systems.

An existing critical problem is that patients frequently have a limited recollection of prior medical health care, the medical health care providers and have no contact information. The patient's recollection or recollection of family members, if any or available, are a poor source for ascertaining a patient's medical history.

The treating clinician does not therefore currently have access to a patient's medical history during a patient encounter, e.g., an emergency room examination. The clinician only has the immediate observation of symptoms to guide treatment. This disclosure allows notation of symptoms and possible diagnosis, immediate suggestion of possible appropriate medical diagnosis, treatment or testing codes for clinician selection, and immediate real-time electronic access (record search and retrieval) to the patient's medical history of past diagnoses, treatments and testing from multiple EHR databases.

Under current practice, records from identified prior health care providers may be manually requested. However, records that are received are often copies or images of paper records with multiple handwritten notations that may be difficult to timely decipher. In other cases, the “records” are summaries of treatment. These summaries may be of limited value to the treating clinician. Also, much of the paper records that are received may be unrelated to the existing medical event and therefore be of little or no value to the subsequent/requesting clinician. Further the record retrieval process may take days to receive. Considerable additional time may be required to parse through the records to ascertain the relevant history. Records obtained from this existing process may be of limited value to the treating clinician having to make real-time decisions and diagnoses for critical patient treatment.

Among the goals of this disclosure is the utilization of comprehensive and industry accepted code protocols, e.g., ICD-10, CPT or HCPCS protocols, to create an index within the patient EHR database. This index may be created simultaneously with the creation of the diagnosis, testing and treatment records. It will be appreciated that these code protocols intended to be adopted into the EHR databases have been authoritatively created. The ICD code (International Classification of Diseases) has been created and is updated by the World Health Organization. Currently the ICD-10 code (replacing ICD-09) is commonly utilized. ICD-11 is scheduled to be implemented in approximately 2023. The CPT® code (Current Procedural Terminology) is authored by the American Medical Association. CPT is a registered trademark of the AMA. The CPT code protocol is updated annually. The HCPCS code (Healthcare Common Procedure Coding System) is based upon the CPT code and is utilized by Medicare for the documentation and payment of medical treatment.

It is a goal of the disclosure to assign one or more relevant codes selected from these protocols to the description of patient diagnosis, treatment or tests recorded in the patient's EHR database. The codes designated in conjunction with medical services recorded in the EHR database can be used as an index to later search the database for urgently needed relevant patient medical history. This assignment of medical codes may efficiently be made at the time such diagnosis, treatment or tests are conducted or entered into the patient's record.

Note that current practice for assigning codes of medical services for approval of third party payment (health insurance or Medicare reimbursement), is conducted long after the patient encounter and diagnosis created. This after the fact correlation of the medical service to the appropriate code occurs by non-medical personal (medical coders) tasked to decipher clinician notations. It may be appreciated that each clinician may utilize its own system or jargon for describing common diagnoses and services. The current use of the code protocols is to “normalize” the descriptions of services for consistent reimbursement. The assignment of codes is performed by medical coders. Medical coders are employed or contracted by the medical health care provider. Frequently, there are mistakes in deciphering the clinician's note and inappropriate codes are assigned. This results in extra time expended by the clinician to clarify the notations, disputed insurance company payment and payment delays.

It is a goal of this disclosure to utilize artificial intelligence or machine learning to enable the clinician to be immediately presented with suggested code options at the time the clinician is making his/her notations during the patient meeting (patient encounter). Various machine learning methodologies may be employed such as key word searching and correlation of notation terms to medical terminology and to a database of code protocols. This can include a correlated database that may be continuously updated by use, i.e., machine learning function. A correlated database receives various terms or jargon used to describe a medical condition. The correlated database allows the correlation of the jargon to medical terminology and the correlation of the terminology to the code descriptions (code descriptors) assigned to each alphanumeric code. The result is that the clinician's jargon, slang or terminology can be converted in real-time into uniform codes of the ICD, CPT or HCPCS code protocols. The methodology becomes streamlined over time and use.

Significantly, utilizing machine learning as part of artificial intelligence, the system may learn to assemble more accurate sets of suggested codes in response to the clinician's text entry. This learning may be based upon prior text entries, search results, correlated database, medical literature and selected codes. The learning could be based not only upon the suggested codes that are selected by the clinician, but the final billing codes entered for the patient's health care event. The goal is to create a common vocabulary and definition of terms between the clinician and the code descriptors of a code protocol.

It is a goal of this disclosure to adopt the code selected by the clinician into the patient's records, thereby becoming part of the patient's EHR.

It is a goal of this disclosure to create electronically searchable and information retrievable EHR databases searchable by individual patient identifiers and indexed by one or more of the code protocols.

It is yet a further goal of this disclosure to enable the clinician to initiate an electronic search of the patient's EHR databases for similar or identical codes contained in the records of past treatment.

It is a goal of this disclosure to have the clinician's electronic search request to be communicated to the various disparate databases comprising the patient's EHR. This communication may be through a network or Internet. It is also a goal to have the records of medical diagnoses, treatments or tests identified by the clinician selected code(s) be electronically returned or shared with the requesting clinician for display.

It will be appreciated that such initiated searches must be incompliance with the Health Information Portability and Accountability Act (HIPAA). The Health Insurance Portability and Accountability Act (HIPAA) does not require a patient's consent be obtained by a health care provider for disclosure of records for continuing medical treatment. Therefore consent should not be required to allow the entity having custody of the past EHR records to furnish requested information to the current health care provider. However, the database or database custodian may want to confirm that the information is being requested for the purpose of furnishing health care services to the patient. This may be part of the database information retrieval process. (It will be appreciated that specific authorization is required by HIPAA to be obtained from the patient to disclose the information for purposes other than treatment, billing or insurance purposes.)

It is a goal of this disclosure that the database search results be available to the clinician concurrent with the patient encounter. This may be termed “real-time” and performed entirely without human intervention. The database subject of this disclosure will return relevant search results to the clinician, e.g., an emergency room doctor, in real time. (By “real-time” or “on the fly”, the Applicant intends a time lapse of only seconds or minutes. The Applicant's use of the term “relevant search results” will be appreciated to mean that a search using codes correlated to symptoms of shortness of breath and chest pain will likely not return information regarding the patient's prior ankle surgery. By “contemporaneous”, the event will take place concurrent with or shortly after the event such as contemporaneous with a patient encounter may mean during the pendency of the encounter or shortly after but before entry of a final diagnosis or treatment plan.)

It is a goal that the EHR database return records containing the class or subclass codes of the clinician selected code. Stated differently, the alphanumeric codes may be organized into child-parent relationships or genus/species relationships. It is a goal that the search request will retrieve records of medical services that have been assigned to such related codes. This will allow a more comprehensive search and retrieval of relevant patient records, i.e., the search will be broader than merely searching for the specific codes selected by the clinician.

It is a goal that the EHR database return records correlated to treatment or tests consistent with or related to a diagnosis or other codes selected by the clinician. This may achieve a broadened search useful to the clinician in verifying if the considered or draft diagnosis is consistent with the patient's history.

In one embodiment, it is the goal that the current clinician can receive the information of past (and alphanumeric code indexed) medical examination, etc., in real time during, for instance, the current initial examination of the patient. The electronic records of the database can be electronically recognized, parsed and the responsive portions be communicated to the requesting clinician without human intervention.

It is a goal of the disclosure that a clinician can be identified to the database as preauthorized to receive patient EHR records from the database. This preauthorization may require identification of the clinician's professional or employment affiliation, e.g., an employee of a recognized health care provider. This goal may verify that the information is being sought for ongoing treatment of the patient and therefore exempt from patient consent requirements.

In an embodiment, this goal of preauthorization may be met by prior consent being furnished by the patient, perhaps through enrollment in health insurance, for the sharing of information from the database for future medical treatment.

An additional goal of this disclosure is to retrieve records that have been assigned to a differing code protocol such as CPT codes that are equivalent to the ICD code protocol or pertain to treatments or tests that pertain to diagnosis codes of the ICD code protocol.

Another goal of the disclosure is enable the search to “cross walk” to older versions of a code protocol. It will be appreciated that the code protocol are continuously updated to reflect changing technology etc. Medical services that were assigned ICD-9 codes should be recognized in a search request utilizing ICD-10 codes.

As yet a further goal, the database may employ cryptography such as public and private keys to allow the clinician to rely upon the authenticity of the received records. Similarly, cryptography can be used to ensure the requesting clinician is authorized to receive the information extracted from the EHR. The validation measures may include block chain technology to ensure the validity and authenticity of the extract portion of the patient's EHR.

This disclosure includes teaching a patient specific EHR database for providing immediate patient medical history and information at the onset of a patient encounter. The immediate access to this information will be invaluable, particularly in an emergency room setting where the health care provide likely has no knowledge of the patient's medical history. It will be appreciated that the health care provider can observe only the patient's symptoms. Critical time must be spent on examination and testing or examination procedures (e.g. X-rays or imaging) before a picture of the underlying causes can be assessed. Under the teachings of this disclosure, past X-ray images may be immediately be made available to the clinician.

### BRIEF SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1 illustrates one embodiment of the database search cycle beginning with the patient encounter and selection of suggested alphanumeric code correlated to a patient medical condition, initiation of a database search request and ending with retrieval of relevant past medical records from the database.

FIG. 2 illustrates variation wherein the search includes review of health care facility billing information for the identified patient and utilizing codes contained in the invoicing records, matching the information to diagnosis, treatment and test procedures recorded in the database.

FIG. 3 illustrates utilization of database access display or search request. Such request is part of the database software management program that correlates the clinician notes into relevant alphanumeric codes consistent with a code protocol which can be utilized for real-time searching of the database and return of the relevant records to the requesting clinician via the search portal. Also illustrated is the function of creating the database subject of the disclosure utilizing or adopting the code protocols into the patient history as clinician diagnosis, treatment and test plans are created and test and treatments notes are entered.

FIG. 4 illustrates an embodiment of the data entry display and concurrent display of suggested codes correlated to the entered medical condition. Also included is patient identifier information, as well as optional information regarding the clinician or health care entity authorization to receive information from the database.

FIG. 5 illustrates a variation of the method and structure for searching the database including steps to correlate selected codes to genus/specie code variations, codes of differing protocols and cross-walking of codes across prior versions.

### DETAILED DESCRIPTION OF DISCLOSURE

It will be appreciated that this Detailed Description of Disclosure includes the above Statement of Use, Brief Summary of Disclosure, and Brief Summary of Drawings. Millions of individuals are examined, diagnosed and treated for medical issues each year in the United States. Each physician, hospital, clinic, laboratory or medical testing facility (“health care provider”) records the examination, diagnosis and treatment pertaining to an individual patient resulting in the daily creation of thousands of individual records that are retained in one or more databases. The records may be maintained by individual patient identity, i.e., the records of the database are patient specific.

These databases are critically important to the documentation of patient health and treatments over time. These databases, comprising the patient EHR, are critical to documenting the patient's healthcare history. A patient's healthcare history is a critical factor in understanding a patient's current malady.

It will therefore be appreciated that ready access to the database of a patient's healthcare history is critical to diagnosing and treating a current health condition. More important is access to information within the database history that is relevant to the currently observed health issue. This avoids time being wasted in parsing through patient medical records or information that is unrelated to the current patient malady. It may also eliminate performance of duplicative, expensive and time consuming tests.

This disclosure creates a novel database having a unique index for each patient's EHR history. The index is established at the time the clinician notations of observed symptoms are entered. The index is supplemented at the time medical procedures are performed or test results entered. The indexing system is not arbitrary but utilizes existing and well-established medical code protocols that have been created by organizations such as the World Health Organization and American Medical Association. These code protocols (now to be used as a searchable index for this critical database) are accepted by the health care industry, health insurance industry and government as providing adequate descriptions and classifications of medical diagnosis, treatment and testing.

The index can be applied and entered as an integral part of the database utilizing machine learning. In one embodiment, machine learning identifies individual clinician jargon or terminology and adopts it to the established coding descriptors. Each descriptor is linked to an individual alphanumeric medical code. In an embodiment, the individual alphanumeric medical code serves as an index entry for the clinician's diagnosis or treatment procedure. The machine learning can utilize medical terminology databases linking jargon to established terminology and to the code descriptors. The machine learning technique improves over time by learning what clinician terminology results in clinician selection of one or more codes of the medical code protocol.

The database indexed by the teaching of this disclosure can also serve as an information source for subsequent searching of the identified patient's medical history. As the clinician identifies symptoms and possible diagnoses, the jargon or terminology can be entered and machine learning causes relevant medical codes to be suggested. The clinician can select one or more of these codes for a search criteria entered into the indexed database. (The database is indexed with this same code protocol.) The database can thereby return relevant historical medical records of past patient history and treatment.

The disclosure may also use machine learning to identify treatment or test procedures likely to be selected by the clinician as a treatment plan. These will be treatments or tests relevant to the clinician's observation of symptoms or preliminary diagnosis. The appropriate codes can be suggested to the clinician. The selected codes can be included in a clinician's search criteria to learn of the identified patient's medical history as contained in the database indexed by the teaching of this disclosure. This expanded search criterial allows the database to achieve an enhanced or supercharged medical history relative to a search of relevant diagnosis codes.

For example, a clinician examining a patient currently being seen in an emergency room complaining of chest pains will have little interest in learning of a past appendectomy, past arthroscopic knee surgery or past tonsillectomy. However it will be critically important to learn whether the patient has had prior treatment for heart disease, heart valve replacement or similar.

It will be appreciated that the patient may not be able to meaningfully communicate with the treating clinician. The patient may be unattended by family. The patient, a resident of Seattle, may have collapsed on a street in Houston.

As taught by this disclosure, the clinician can electronically record the observed medical conditions of the patient and software suggested medical codes (with text code descriptors) can be instantly displayed to the clinician. It will be appreciated that the suggested codes are correlated to the noted symptoms. The clinician can enter the symptoms, etc. in his/her own jargon. The software used in creation of the database can, in one embodiment, employ a correlated database with machine learning. The clinician terms are correlated to medical terms using a machine learned database. The database can contain medical terms derived from published medical literature, treatises, dictionaries, etc. The database can be taught a correlation between common vernacular or jargon and the formal medical terminology. Overtime, machine learning will enhance this correlation. This enhancement may involve learning the use of certain terms of jargon are related to or synonyms of formal medical terms based upon clinician selections over time. The formal medical terminology can be used with or correlated to the code descriptors used with specific alphanumeric codes. This correlated database employs machine learning as the mechanism for continuous updating, i.e., continuously learning. The clinician can evaluate the suggested codes with the text code descriptors, select among the suggested codes and immediately initiate an electronic search of medical databases for the records related to past patient diagnoses, treatments or tests conducted relevant to the noted symptoms (identified by the codes).

The search will identify the patient. The identification can include the patient name, social security number, date of birth (DOB) and other optional identifying information. Information identifying the clinician, clinician professional or entity affiliation or license number, reason for request, patient authorization (if possible) and contact information can be included in the search request.

It is recognized that all records within the databases may not be indexed or otherwise correlated to the codes identified in the search request. In an option, the search request may seek access to healthcare provider medical billing or payment records. This is in recognition that use of the code protocols has been long established for medical cost reimbursement purposes. Stated differently, invoices submitted to third party payors (health insurance companies, Medicare, etc.) will have codes assigned as part of the description for reimbursed medical services, thereby identifying past medical treatments, tests and diagnoses. The identified codes, services, dates of services, etc., can be used to match EHR database records of diagnoses, treatments or tests. The separate databases can therefore be interrelated.

Therefore the billing or reimbursement records can serve as a bridge to access the EHR database that is otherwise not indexed using the code protocols as taught by this disclosure.

As stated above, the disclosure teaches incorporating authoritatively created and industry accepted comprehensive code protocols into patient EHR databases to serve as indexes for future clinician searches of patient history. These code protocols include the ICD series, (ICD-9, ICD-10, ICD-11) CPT code protocol (updated annually) and the HCPCS protocol utilized by Medicare.

### Example

For purposes of illustration only, an example of wherein obtaining a patient's history of past medical conditions could occur with a present clinician observing patient shortness of breath. It will be appreciated that an observation of patient shortness of breath can have multiple causes. Causes may vary from cold, pneumonia, COPD (including emphysema or chronic bronchitis) anxiety, fear, to asthma, CO poisoning, pulmonary embolism, cardiac failure. Using “shortness of breath” as a diagnostic term, software (using machine learning) may return a suggested ICD-10 code of J45—unspecified asthma.

It will be appreciated that the clinician's selection of J45 as a database search term (parent term or genus) would include searches for child or specie code entries including:

J45.2 Mild intermittent asthma

J45.3 Mild persistent asthma

J45.4 Moderate persistent asthma

J45.5 Sever persistent asthma

J45.901 Unspecified with acute exacerbation

J45.902 Unspecified asthma with status asthmaticus

J45.909 Unspecified asthma, uncomplicated

J45.998 Other asthma

J45.90 Unspecified asthma

J45.99 Other asthma

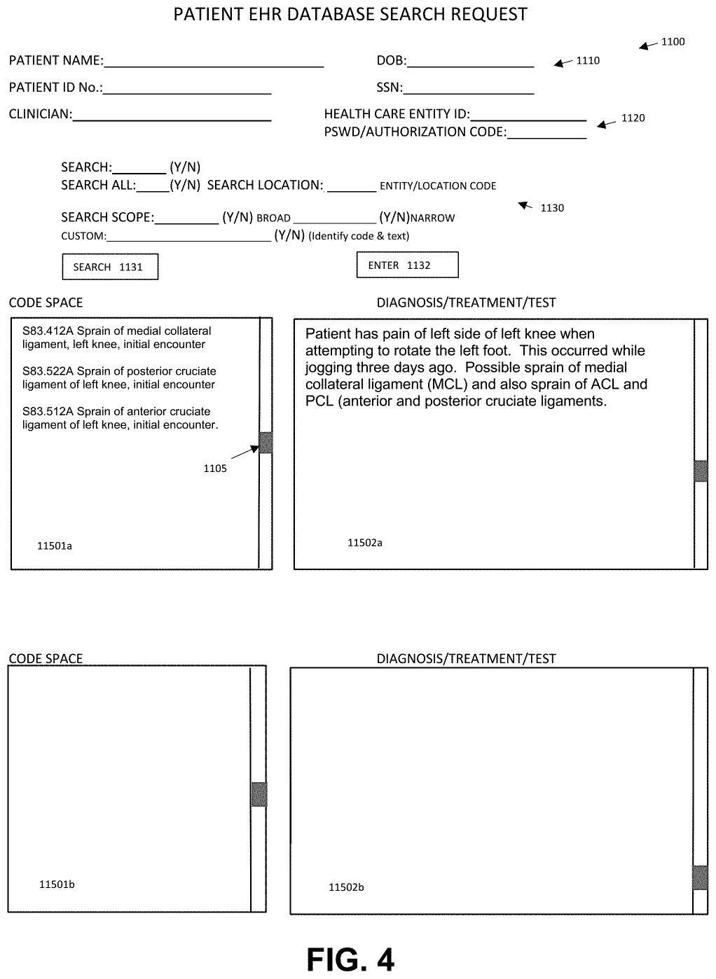

It will be appreciated that other generic codes may be suggested and selected by the clinician for COPD, pulmonary embolism, etc. Such a search will return records from the database for species and subspecies. With reference to FIG. 4 discussed below, the clinician may elect to have a narrow or broad search performed. The disclosure of suggested codes may facilitate the clinician constructing a tailored search.

Similarly, the CPT codes, i.e., primarily procedure and test codes, could be parsed and codes selected for asthma testing and treatment. For example, the software could identify the following exemplary codes, e.g.,
1005F Asthma symptoms evaluated
2015F Asthma impairment assessed
2016F Asthma risk assessed
5250F Asthma discharge plan present
4015F Persistent asthma long term control medication prescribed
1038F Persistent asthma mild moderate or severe asthma
1039F Intermittent Asthma
94375 Respiratory flow volume loop
94640 Pressurized/non-pressurized inhalation treatment
0276T Bronchoscopy, rigid or flexible, including fluoroscopic guidance, when performed; with bronchial thermoplasty, 1 lobe
94728 Airway resistance by impulse oscillometry

Again, this search could be conducted immediately and search results displayed to the clinician in real time to aid in the diagnosis and treatment process. It will be appreciated that the search results may therefore show a patient history of treatment for a malady that displays the symptoms being currently observed by the clinician.

Depending upon the search results, the clinician may initiate one or more follow-up database searches to facilitate assessment of the patient history. The clinician, utilizing the search results, may order tests relative to the past malady. If the test results return negative, the clinician may broaden the scope of the tests.

In regard to possible pulmonary embolism, responsive ICD codes could include but are not limited to the following:

I26 Pulmonary embolism (parent code) and the following child codes:
I26.0 Pulmonary embolism with acute cor pulmonale
I26.01 Septic pulmonary embolism with acute cor pulmonale
I26.02 Saddle embolus of pulmonary artery with acute core pulmonale
I26.09 Other pulmonary embolism with acute cor pulmonale
I26-128 Pulmonary heart disease and diseases of pulmonary circulation

127.9 Pulmonary heart disease unspecified

127.20 Pulmonary hypertension unspecified

127.82 Chronic pulmonary embolism

174.10 Embolism and thrombosis of unspecified parts of aorta

174.11 Embolism and thrombosis of other parts of aorta

286.711 history of pulmonary embolism

CPT codes pertaining to pulmonary embolism may be suggested to the clinician or automatically included within the initiated database search. Class and subclass codes could be included, particularly if the clinician elects to have a broad or narrow search conducted. These CPT codes include but are not limited to:
33910 Pulmonary artery embolectomy with cardiac bypass
75741 Angiography pulmonary unilateral diagnostic radiology procedures of aorta and arteries
75743 Angiography pulmonary bilateral SLCTV RS&I
75746 Angiography pulmonary non-selective CATH/VEN NJX RS&I
33471 Valvotomy pulmonary valve CLSD Heart via pulmonary artery
33475 Replacement pulmonary valve
33917 Repair pulmonary artery stenosis JCNSTJ w/patch/graft
33920 Repair pulmonary atresia w/CONSTJ/RPLCMT conduit
33922 Transection pulmonary artery w/Cardiac bypass

As noted above, one additional cause of shortness of breath can be cardiac failure or similar. The software may display ICD-10 diagnostic codes related to cardiac distress such but not limited to ICD code 150. This code may lead to the following species or child codes:

I50.9 Heart failure unspecified

I50.1 Left ventricular failure unspecified

I50.20 Unspecified systolic (congestive) heart failure

I50.21 Acute systolic (congestive) heart failure

I50.22 Chronic systolic (congestive) heart failure

I50.30 Unspecified diastolic (congestive) heart failure

I50.31 Acute diastolic (congestive) heart failure

I50.32 Chronic diastolic (congestive) heart failure

I50.89 Other heart failure

I50.82 Biventricular heart failure

I50.810 Right heart failure unspecified

I50.814 Right heart failure due to left heart failure

I51.5 Myocardial degeneration

I151.9 Heart disease unspecified
It will be appreciated that a broad search for code 150 will be productive with the knowledge that database records pertaining to the child codes, e.g., 150.9 heart failure unspecified, will be returned.

It will be appreciated that this is only a partial list of diagnostic codes that may be prompted by a clinician's notation of the symptom of shortness of breath related to a possible heart condition. As already shown above, treatment or test codes (e.g., CPT codes) will also be generated for the rapid, real-time electronic search of the patient's EHR that may be initiated by the clinician. In an embodiment, the scope and order of test and treatment codes may be a product of machine learning evaluation of the noted symptoms.

### Example

As another example, an orthopedic surgeon evaluating treating a patient for complaints of left knee pain may be interested in records pertaining to past left knee orthoscopic surgery. The orthopedic surgeon may not be interested in medical records pertaining to the patient's past hospitalization for pneumonia. In this situation, it would be productive and time and cost efficient to request medical records from the database for patient that pertain past diagnosis, treatment or testing (e.g., X-rays) to patient's left knee. This disclosure teaches a method for prompt receipt of relevant EHR database information pertaining to the left knee by requesting the EHR by the patient's identification and the relevant diagnosis and treatment codes. Machine learning may also cause codes for the leg, ankle or foot to be displayed. It will be appreciated that past ankle or foot surgery, therapy, etc., may be relevant to the present complained left knee pain. The codes utilized for billing purposes also have an expanded utility of prompt and efficient receipt by a clinician of past diagnoses and treatment.

The search request could specify records associated with particular billing codes for knee surgery, X-ray or other imaging of the left knee, pharmacologic treatment for inflammation or treatment of arthritis. In one embodiment, the disclosure contemplates treatment and test codes relevant to the clinician's diagnosis will form part of the database search. This enhanced searching utilizes artificial intelligence and machine learning to select relevant treatment and test codes. It will be appreciated that this embodiment will cast a broader net for searching of relevant records. Again, and with reference to FIG. 4, the clinician may select the breadth of the database search, including the search to a particular code. The search may not be limited to the clinician's adopted diagnosis codes.

It is worth repeating that this disclosure can be expanded to allow indexed database searching of the EHR database for past treatment and tests conducted on the patient. The search request can include all relevant records correlated to the generic diagnosis code, e.g., J45 for unspecified asthma. This can return X-ray images, as well as lung capacity testing or drug therapy. It may disclose related and current medications prescribed for the patient.

It will be further appreciated that this disclosure teaches novel expansion or use of coding protocols currently principally utilized solely as a billing coding protocol and payment mechanism. It also expands utilization of codes as a tool to search voluminous databases in real-time for not only similar diagnosis but treatment and test. This will be of great usefulness for the clinician. Knowing the patient history will allow treatment of the underlying causes in contrast to merely treating observed symptoms.

Also, current adoption of annotating or indexing each clinician's record of diagnosis and treatment with the CPT or ICD codes, now, allows a later clinician expedited and efficient access to patient's database of medical records. It will also be appreciated that current coding typically occurs after the fact (hours or days after the patient encounter) and is performed by medical coders, in contrast to a clinician interacting with a patient.

As another example, the teaching of this disclosure is also invaluable in the admitted rare but real exigent circumstance where a scarce life saving medical resource must be allocated among competing patients. For example, information disclosed by the database taught by this disclosure and regarding the patient's medical history, e.g., history of respiratory condition and disease, may be relevant in determining whether an individual patient should receive use of a ventilator when another patient, also requiring a ventilator, does not have an adverse medical history. Sadly this is a real occurrence at the time of submission of this disclosure.

One aspect of this disclosure is to provide diagnostic software operated by the database search engine or portal to expand the patient health records created by one or several health care providers into a more comprehensive indexed electronic health record (EHR) by encouraging the common use of medical coding schemes or protocol such as CPT that is applied to all patient records created at all (or nearly all) heath care providers. A further goal of this disclosure is to have all EMR created and maintained by separate health care entities accessible to clinicians via system wide assessable EHR database. Stated differently, a clinician examining a patient in Houston will be able to have real-time, immediate access to the patient's medical records of prior treatment in Seattle.

Further, the current practice of utilizing the coding protocols solely for invoicing purposes may ignore specific diagnosis and treatment steps but rather be edited by an entity's, e.g., hospital, coding assignment and review procedures to reflect a general “theme of the patient treatment.” A clinician's diagnostic notes of codes may be omitted from the EHR database thereby causing the database search to be incomplete. These omitted clinician notes may contain nuanced information that will be valuable for a later separate clinician treating the same patient. The clinician's adoption of the coding protocol, as assisted by the software and display devices utilized with this disclosure and in contrast to the medical coder's interpretation, can provide the later clinician with complete access to the patient's EHR (wherever located). As stated above, the coding protocol can be used as an index to search a patient's EHR database.

The patient diagnosis and treatments records contained within the individualized EHR can be indexed by patient identity, e.g., name, SSN, service provider assigned patient number, insurance provider and insurance policy number, etc.

Indexing Clinician Notes into EHR Database, Adoption of Codes and Focused Searching

This disclosure may be utilized with a software tool that allows electronic records of a clinician's diagnostic or treatment notes to be easily and rapidly incorporated into the patient's EHR database. Utilizing machine learning, the software displays suggested codes deemed possibly relevant to the clinician entered symptoms. The clinician can accept all or some of the suggested codes or modify his/her description of symptoms. Accepted diagnostic and treatment coding protocols correlated to the clinician's notes can be entered as part of the clinician's entry or creation of an EHR record. The record becomes part of the patient's EHR database. The database is indexed and later searchable by this code index. The correlation can be performed by continuously updated correlated database utilizing machine learning. This updating is part of the software of this disclosure.

As stated, the codes may be adapted by the clinician. This display and adoption of codes into the patient's EHR occurs at the time of the patient encounter. Again, these incorporated codes create an index of the patient's medical records by the coding numbers that is adopted by the clinician. This indexing, created by the use of the software, will allow a medical service provider to search and receive past patient's treatment records by the service (diagnosis, treatment, laboratory test results, etc.). The relevant past medical service records are accessed by utilization of the uniform service coding protocol. The relevant past medical records can be accessed via this diagnostic and treatment software. See FIG. 4.

The novel features of this disclosure include the coding protocol, utilized to facilitate medical payment, expanded to be used to create an indexed and searchable EHR database of individual patient EHR medical records. Search by medical treatment code numbers would substantially narrow the scope of review required of health care providers. It would also save time and effort of the health care providers in searching and copying records.

The present health care provider may have greater confidence in the accuracy of database records received from individual past providers, in contrast to receiving redacted records from one or more central clearing house or third party service. The present clinician may have greater confidence in records from a database utilizing authentication tools for data entry and retrieval

In contrast with utilization of patient identity information with specific indexing codes, a request medical records solely by patient name or SSN will result in a totality of all medical records i.e., EHR, pertaining to that patient being provided. However, the party requesting the records (the requestor being another medical clinician providing current medical services related to a past medical treatment) may be interested only in medical records pertaining to a specific medical condition, e.g., shortness of breath.

In one embodiment, a search request may comprise the patient's name, date of birth and possibly other identifying information such as social security number, address, date of past services (if known) and a consent for release of information signed by the patient. (This consent can be obtained as part of the patient's initial information disclosure to the health care entity, e.g., clinic or hospital. This patient information disclosure may be part of the EHR records stored in the database.) The search request would also describe the instant treatment issues and the code or codes associated with that medical issue. The database recipient of the search request may only be required to electronically locate the records, and submit records responsive to the listed coding information. This can be performed instantly without human intervention. It of course will appreciated that the full EHR may be later submitted if requested.

It will be appreciated that over time, an individual patient's EHR will increase in size as the patient receives additional medical examinations and treatments for multiple medical events or conditions. The number or content of medical records indexed by the teaching of this disclosure will also increase. It will further be appreciated that the subject matter of the treatments will vary. It is not time or cost efficient for the voluminous totality of past medical treatment records (PHR) be solicited from multiple service providers and then parsed for information pertaining to the instant medical event. Even if the patient's records are at one location, it is not feasible to quickly parse through voluminous records that pertain to multiple diagnoses and treatment to find information specific to the instant illness or trauma. The automated indexing taught by this disclosure and focused search and retrieval methodology eliminates this effort. It will be appreciated that the teaching of this disclosure addresses a serious problem in the administration of health care to individuals and supplies a novel and reliable means to solve this problem.

Under current practice, the providing of medical services may include the steps of (i) patient identification, (ii) identification of patient payment mechanism for medical services, (iii) examination, (iv) diagnosis (v) treatment (vi) submission of records for coding, (v) submission invoice to third party payor for payment, and (vii) record retention. There may also be the step of adding the records to the totality of the EHR. The retained records are not, under current practice, correlated to the assigned payment coding. However, the payment coding scheme or protocol is intended to have a comprehensive number set for all diagnoses, treatment, surgery, drug therapy and ancillary services such as X-ray, test scans (e.g., MRI) and laboratory work and testing. This disclosure teaches an expanded use of the ICD or CPT codes from merely a tool for classifying treatment and diagnosis for billing but to provide a comprehensive index of medical treatment. This comprehensive index solves the problem of a treating clinician receiving the relevant portion of a patient's medical history in real-time. Patient diagnosing, commencement of treatment and testing will be greatly enhanced.

As used herein, “patient electronic medical records” (or EMR) or “electronic health records” (or EHR) comprise multiple (and separately located) databases and include, but are not limited to, machine readable electronically stored records incorporating ICD, CPT or HCPCS code protocols. The EMR/EHR records contain the recorded text of the health care provider's observations of the patient, patient history and diagnosis, as well as treatment notes, record of ordered tests and test results. It may also contain images, e.g., X-rays and CT scans.

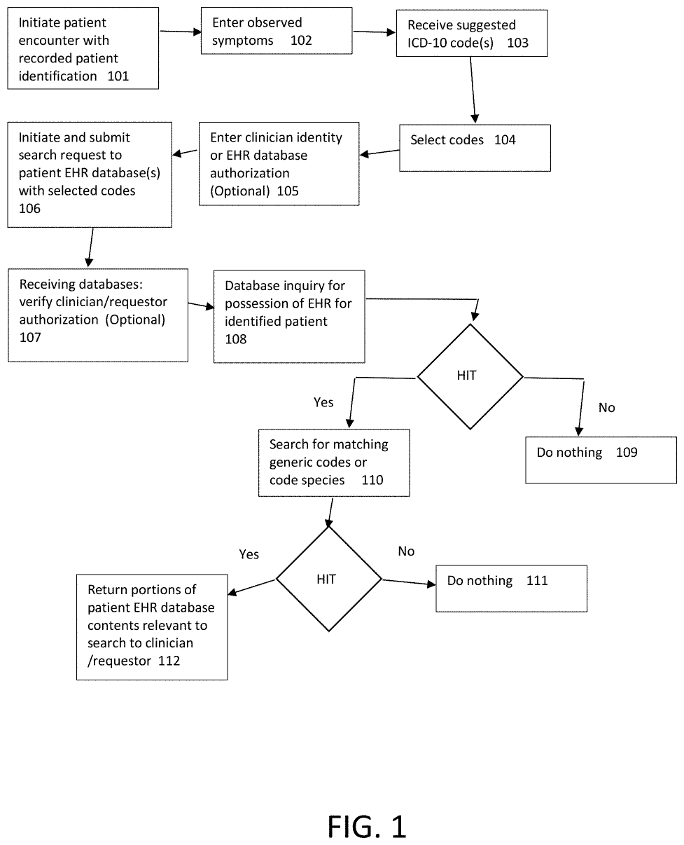

Attention is drawn to FIG. 1 of the disclosure. FIG. 1 provides an overview of the database operation including the search and retrieval function. Utilization of the database begins with the patient encounter 101, data entry of observed symptoms of the patient medical condition 102 and receipt and display of codes suggested from one or more medical code protocols, e.g., ICD-10, 103. The clinician may select among the suggested codes 104 for initiation of database searching. These may be separately located databases. (It will be appreciated that the clinician may amend or supplement the entry of observed symptoms or preliminary diagnosis based upon the contents of the suggested codes.) Note the patient identity has already been disclosed. See FIG. 4. There may be an optional step for confirming the authorization or authority of the requesting party, e.g., clinician or health care provider 105. This may be required to achieve access to one or more databases.

The database (containing EHR records of multiple individuals collected over time) is contacted by the clinician's search request 106. Communication may be by means of an electronic network or Internet. Access may require utilization of one or more application program interfaces (API). The search request identifies a specific individual, i.e., the clinician's patient. Various identifiers may be used. The database may require confirmation of the identity of the clinician or health care provider 107. This may be a step for maintaining confidentiality of the records contained within the database. In addition to the identifying the patient, the search request contains at least one alphanumeric code, e.g., an ICD-10 code. This search functionality, e.g., a search engine component of the database, need only search the database for the existence of records pertaining to the identified patient. In one embodiment, the database may communicate to the initiating party that the search request has been received. The database may also communicate if the authentication/access requirements have been unsuccessful, requiring a repeat of the process or request further information (passwords, security questions, etc.).

In an embodiment, the database searches for the existence of records pertaining to the identified patient 108. If there is no match or “hit”, the process can end at that point 109. The database may communicate back to the requesting party that no records for the identified patient have been located.

In an embodiment, the search functionality will inquire of records pertaining to the located patient/individual that contain the alphanumeric code 110. If records are located, the records pertaining to the identified code will be retrieved for communication to the requesting party 112. In an embodiment, the contents of these records may include the records of the earlier patient encounter, including the prior clinician's notes of symptoms, etc. Included may be diagnoses, treatment and test results and discharge summary. Of course, if no records responsive medical records (such as indexed records) are located 111, there will be no records to communicate to the requesting party. This absence of responsive records may be communicated to the requesting party. The clinician may initiate a repeat search of the databases with different or broader search request, e.g., with different selected codes in response to revised clinician input.

It will be appreciated that in an embodiment, the search may not be limited to the specific alphanumeric code selected by the clinician but rather to related codes such as other parent/child codes or genus/species codes. It will be appreciated that this function (broadening the scope beyond the specific alphanumeric codes specified by the clinician) may be performed by software of the search request process. Alternatively, it may be performed by the database or database search engine. This intelligent search functionality may be a part of the EHR database structure and function.

### Example

In one embodiment, the search functionality contains instructions and non-volatile memory comprising at least one code protocol. The memory includes organization of the code protocol identifying parent/child or genus/species relationship among the various codes. Thus the clinician's listing of code I50.30 (Unspecified diastolic (congestive) heart failure) will automatically trigger a search of multiple codes within that class 150 or subclass (parent/child or genus/species), i.e., I50.9 (Heart failure unspecified), I50.1 (Left ventricular failure unspecified), I50.20 (Unspecified systolic (congestive) heart failure), I50.21 (Acute systolic (congestive) heart failure), etc.

### Example

In an embodiment, the database structure will group or compile records associated with one or more alphanumeric codes. Therefore it will not be necessary for search time being expended to ascertain what records are to be communicated to the requesting party. This will speed up the communication between the database and requestor.

It will be appreciated that one goal of the disclosure is for the search and response to be conducted without delay and in real-time. For example, the search can be initiated by a clinician during a patient encounter and the search results communicated for display to the clinician before completion of the patient encounter. This will facilitate proper diagnosis and creation of an effective treatment and test plan. This may cause initiation of additional questions to the patient regarding symptoms or past treatment.

It will be appreciated that records communicated from the database may include X-ray images or similar prior test results.

It will be further appreciated that the EHR database, constructed and organized for purposes including the search and retrieval of patient records in response to later requests associated with patient treatment, may be equipped with search functionality capable of correlating diagnosis codes, e.g., ICD-10 codes, with treatment or testing codes, e.g., CPT codes. Accordingly, a search request including an ICD code within class 150 (indicative of a heart condition) will also trigger a search of probable treatment and test codes such as CPT codes. This function will broaden the scope of the search and return patient database records to the clinician of past test results, etc., that may be of great value in creation of an accurate diagnosis and treatment plan. This will be a supercharged database. This supercharged search can be activated through a software display accessed by the clinician. See FIG. 4.

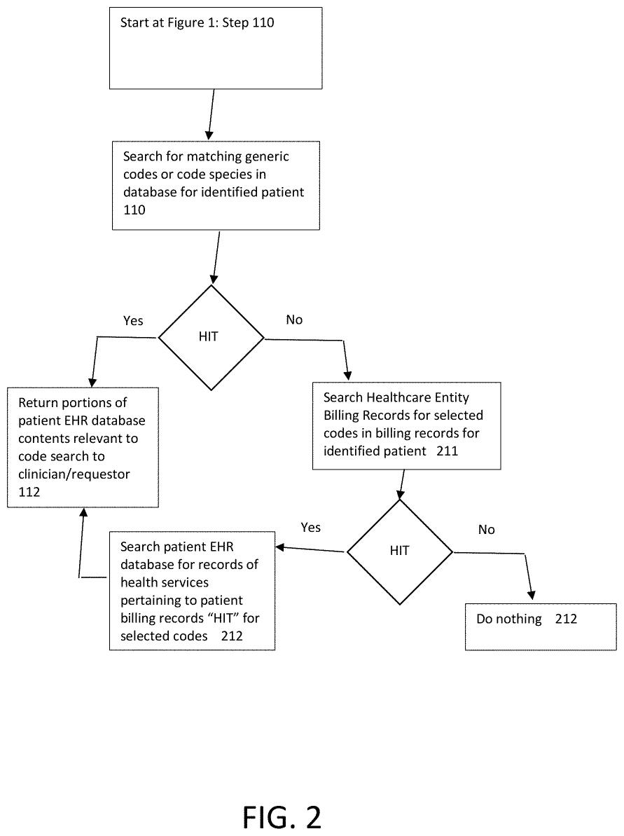

FIG. 2 illustrates an embodiment wherein a database can provide records notwithstanding the database not being indexed or organized with alphanumeric codes from a code protocol. This may be relevant for databases created prior to the practice, taught by this disclosure, of adopting the code protocols into a patient EHR records stored in the database.

FIG. 2 illustrates the situation wherein the database contains records pertaining to the identified patient but does not have documents referencing a code protocol 110. In that situation, it may be possible for the search be directed to a health care provider's billing records 211. It will be recalled that the code protocol have a long and established use as an integral part of a health care provider's invoicing practices. Therefore examination of the billing records will reveal past medical treatment or procedures that have been indexed to the subject medical code protocol, e.g. ICD-09, ICD-10, CPT or HCPCS, 212. Dates of relevant services can be identified which can be used for a supplementary database search 112. Therefore the billing records can be used as a bridge or alternate pathway to the patient EHR medical database records or creation of an indexed database.

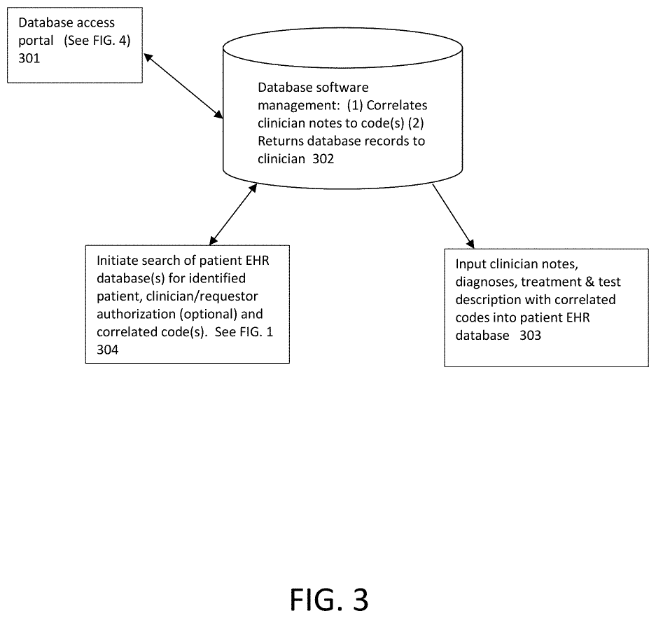

FIG. 3 illustrates use of a database portal 301. Reference is made to FIG. 4 1100 wherein the patient identifiers used by the database are inputted 1110. See FIG. 4. In an embodiment, the disclosure teaches database software management that correlates the clinician's input 11502a with a medical code protocol 11501a. See FIG. 3, 302. This database software management also makes the retrieved database records available for display to the requesting clinician. Also illustrated is the database search, including option authentication of the requestor/clinician and the correlated codes 304. It will be appreciated that the database software management inputs the diagnosis, treatment and test records of a patient encounter and treatment (along with the correlated alphanumeric codes) to the database 303. This step provides an updated organized or indexed database available for later searches and data retrieval.

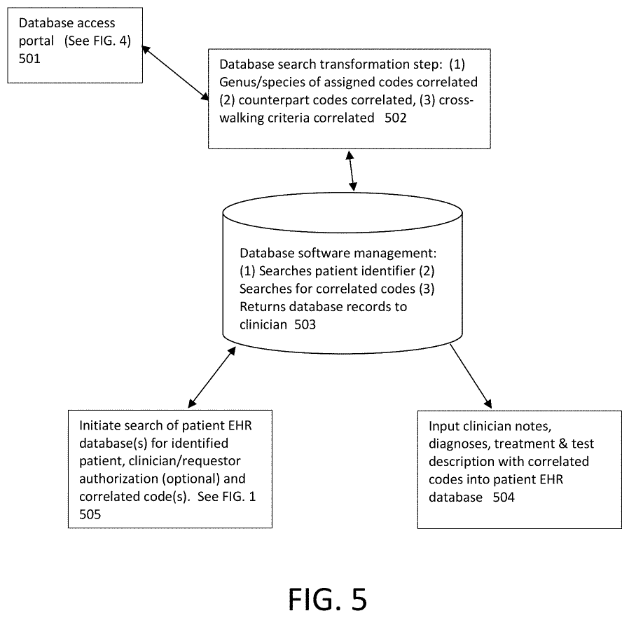

FIG. 5 illustrates an alternative embodiment wherein the software operating the database portal performs the database search transformation step 502 of identifying the code classification, i.e., class/subclass, genus/species or parent/child classes, identification of counterpart alphanumeric codes, i.e., ICD-9, ICD-10, CPT or HCPCS, as well as associated codes for diagnosis (ICD) and treatment or tests (CPT or HCPCS). Also included is cross-walking, i.e., correlating a present ICD-10 code to a prior coded ICD-09 entry. The database search, in other embodiments, can perform these tasks, i.e., the “supercharged” database. FIG. 5 also illustrates the database performing the search for a patient identifier, i.e., whether the database have any records for the patient/individual identified in the search request 503. If yes, the search functionality can retrieve the correlated codes and communicate the records to the requesting clinician. Note this step incorporates the results of the search outlined 505. The search functionality or alternate subcomponents may organize the input of information entering the database 504.

Returning to FIG. 4, the database search request or portal 1100, the database request form includes information requests 1120 that can be used to identify and authenticate the clinician or requesting party. This can include information concerning the health care facility employing the clinician or affiliated with its facilities. A clinician or health care provider may be “pre-cleared” to receive data from the database records.

The database search request can also allow the requesting party to tailor or designate the breadth of the requested search 1130. This may allow an abbreviated search that will return search results in a shorter amount of time. The search request may be customized to identify a particular record pertaining to the patient. Note also that the search request can allow the clinician to enter the observation of symptoms or diagnosis 11502a, along with selected suggested codes 11501a, into the database. It will appreciated that selection of one of the suggested codes initially appearing may cause the non-selected suggested codes to be automatically or manually removed from the display 11501a. This will clarify only clinician selected codes will be searched. It will also be appreciated of course that there are multiple databases that can be subject of the search request. The database search request, which can be a display on an electronic device utilized by the clinician, can initiate the search 1131 or entry of data 1132 with a single click. The display device can incorporate scroll bars 1105 to allow additional information to be displayed via the display boxes or screens. The clinician may enter observation notes in one display screen 11502a and subsequent test results or treatment plans in a second screen 11502b. It will be appreciated that additional screens can be displayed using a scroll device or similar functional device (multiple page selections).

Accordingly, this disclosure teaches a system for improving the efficiency and reliability of a patient's EHR database that may be utilized by the clinician in contrast to creating a database search request by merely entering search words. The database subject of this disclosure is receptive to search requests incorporating ICD, CPT etc., codes or completions to the data entry operator, e.g., clinician. The entries can include, but are not limited to, alpha-numeric, alpha or numeric codes contained in a medical provider service code protocol. The operator/clinician enters data regarding the description of diagnosis, treatment or testing into a particular data area and a dynamic list of possible codes is generated based on entered text for the diagnosis, treatment or testing, etc. The entry of text may be based upon inclusion of key words or the analysis by the software (machine learning function) of the entered text. See the suggested software screen display for data entry presented in FIG. 4.

The CPT treatment codes, prompted by the device software, i.e., the codes correctly describing the treatment/test protocol selected by the clinician, are included within the patient history. Again it will be appreciated that this embodiment allows the correct code to be selected concurrent with treatment and testing and not “second guessed” by a later medical coder.

This aspect of the disclosure frees the clinician from attempting to memorize the applicable codes or search a code index to select the appropriate code. This accelerates the data entry process and provides greater accuracy in entering the correct code for the services, treatment or diagnosis, as well as entering of the description of service or treatment.

As suggested above, in another embodiment, the algorithm may suggest treatment or test and related codes in response to the clinician's symptom or diagnosis entry. This will provide a more complete or robust search, again casting a wider net for a search of the patient's EHR history. This is an example of the super-charged database.

The display may contain a “Search” button 1131 to initiate a search of the patient's EHR. It may also include a button or other mechanism (not shown) to reject a suggested code. Acceptance of the suggested code may cause the entry to replace the health care provider's notes or to be included within the accepted/integrated code protocol entered into the EHR. It will be appreciated that the latter option will preserve the clinician's notes (and the nuance that may be contained therein) for the benefit of later clinicians retrieving such notations as part of an EHR search taught by this disclosure.

FIG. 4 illustrates a further embodiment of the display screen 1100. The patient information is entered and displayed 1110. Also is the identity of the clinician user 1120. This information may be utilized as part of the authentication steps discussed below. Optionally, the screen display may provide input 1130 regarding the scope of any initiated search. Again, this may be a decision made by the clinician in the interest and urgency of time. The display provides for a “one click” initiation of a search 1131. Also there is the one click initiation to accept entered and suggested data into the EHR database 1132. It will be appreciated that the software display of FIG. 4 is in communication with the EHR database of at least one health care provider.

Initiation of the search will transmit the search request, including patient identifying information (and optionally clinician or health care provider identifying information or authorization) to at least one database via a network or Internet. The system subject of this disclosure may include not only the program for entering the clinician data, as well as a correlated database or other means for converting or assigning codes responsive to the clinician's input, but also network connections such application program interfaces to access at least one database. The database will have capability to receive the search request, database search functionality, e.g., search engine, and initiate data transmission back to the requestor.

In regard to the clinician being able to select a “broad” or “narrow” search is the goal that the suggested codes include the generic code as well as the species that may be selected by the clinician (or modified by amendment of the code descriptor or correlated database). For example, a code indexed diagnosis for pain in knee may include broad generic codes for knee (as well as the leg, ankle or foot) as well as one or more species codes for specific to the clinician's diagnosis. The device subject of this disclosure may allow the health care provider to tailor a search broadly or narrowly. For example, a diagnosis of pain in the patient's right knee may include: “M25.561 Pain in right knee”. However, in one embodiment, the clinician may request a broader search, to include the code: “M25.56 Pain in knee”.

FIG. 4 also illustrates are “scroll bars” 1105 or scrolling features (controlled by finger “swiping”) to allow greater information content to be accessible to user via the screen. In an embodiment, the clinician may elect to have displayed only segments of information such as code space 1101a or Diagnosis/Treatment/Test 1102a. Again, this selection feature may be controlled by “swiping”. Also it will be appreciated that this feature may be most useful when a mobile device, having limited display screen space, is being utilized.

In another example, and referencing FIG. 4 discussed below, the clinician may enter the class descriptor ICD-10-CM S83 Dislocation and sprain of joints and ligaments of knee. This class descriptor correlates to the suggested subclasses S83.412A, S83.522A and S83.512A.

In yet another embodiment, the clinician may request that the listed medical code entries of an ICD-10 or CPT code protocol be correlated to include similar code entries of at least one of the following alternate codes protocols, Healthcare Common Procedure Coding System (HCPCS), Diagnostic Related Grouping (DRG) codes, etc.

Using the identified data, i.e., the EHR entry, the code and the code classifier/descriptor, the text of the EHR entry may be classified by the algorithm of the system into one or more of the classes or subclasses. It will be appreciated that this will enhance the returns in subsequent searches.

### EHR Search Request—Search Request Initiation

FIG. 4 illustrates another embodiment of the disclosure whereby access is obtained to the database via a software generated display screen 1100. The request for a record search may be activated by entering the information prompted on the display screen 1100 indicated on the data entry form and pressing the search key 1131 of the user input component 1120. In other embodiments, one or more radio buttons may be used. The patient identification information may be entered 1110. Also the clinician entering the data may be identified 1120. The clinician may be subject to preauthorization or identification. This authorization or identification standards may be subject of prior implemented procedures.

Note that the device illustrated in FIG. 4 allows the user to initiate a search 1131 of the patient specific EHR. The scope of the search may be specified to be broad 1130, wherein perhaps all related search codes or search words may be use as supplemented by machine learning or the search be customized or limited to certain key phrases or codes. The contents of the display screen may be entered or integrated into the patient specific EHR by activation of the “enter” key 1132.

Note further that in one embodiment of the device 1100, each diagnosis/treatment/test data entry space 1102a, 1102b and each code space 1101a, 1101b contains a scroll bar 1105 or similar device that allow entry and view of content within a space larger than the specific size of the screen display, i.e., 1102a.

Several different methods will be used to allow the clinician to pinpoint what code(s) to apply to the patient encounter for enhanced diagnosis and real-time EHR record searching:
Medical Term Match—A match/find of ICD Codes by a mapping of those medical terms (as keywords) that are found in the description of an ICD Code, i.e., code descriptors.
Patient Encounter Synonyms (current)—Code matching through keyword extraction (current encounter)
Patient Encounter Synonyms (previous)—Code matching through keyword extraction (previous encounter(s))
Entity-wide Encounter Synonyms—Code matching through keyword extraction of a table of codes from encounters held across the system, not just those from the specific patient's encounters. It will be appreciated that this step creates more robust search since the disclosure is making the query of what codes have been assigned to similar diagnoses or medical conditions, i.e., patient agnostic. This table may be akin to the correlated database that is continuously revised as part of the machine learning function.
Invoice Match—Code matching against code used in previous invoices of the current patient. (Has the patient been treated for this or similar condition in the past as determined from the invoice records? Again this creates a more robust search.)
EHR Match—Code matching in previous Electronic Health Records (or electronic medical records, i.e., EMR) of a patient. This can use input from the Invoice Match. This can be useful when the code protocols have not been incorporated in past EHR entries. The invoice description of services, combined with the code protocol contained in the invoice, can substitute for a code indexed EHR.

This specification is to be construed as illustrative only and is for the purpose of teaching those skilled in the art the manner of carrying out the disclosure. It is to be understood that the forms of the disclosure herein shown and described are to be taken as the presently preferred embodiments. As already stated, various changes may be made in the shape, size and arrangement of components of the system or device. Also adjustments made in the steps or order of the steps of the method without departing from the scope of this disclosure. For example, equivalent elements may be substituted for those illustrated and described herein and certain features of the disclosure may be utilized independently of the use of other features, all as would be apparent to one skilled in the art after having the benefit of this description of the disclosure.

While specific embodiments have been illustrated and described, numerous modifications are possible without departing from the spirit of the disclosure, and the scope of protection is only limited by the scope of the accompanying claims.

What we claim:

1. A method of rapid retrieval of relevant patient records of past medical diagnoses, treatments or testing by indexing patient EHR medical records created in a past patient encounter and retained in an EHR database wherein the records creation includes integrating and adopting recognized medical coding protocols into the patient diagnosis, treatment and test records comprising:
(a) inputting a patient identifier and patient medical condition during a patient encounter into an electronic transitory or non-transitory memory record;
(b) utilizing machine learning to assign in real-time at least one code correlated to the patient medical condition noted in the patient encounter wherein the code is selected from a protocol of medical codes;
(c) submitting an electronic search request via a network or Internet to an EHR database containing patient specific records wherein the search request contains the patient identifier and the at least one code; and
(d) requesting an electronic search of the EHR database having a non-transitory memory containing records of past medical diagnosis, treatment or test for the patient relevant to the at least one code included in the electronic search and return transmission of responsive records contemporaneous with the patient encounter to facilitate the diagnosis, treatment or testing of the patient medical condition.

2. The method of claim 1 wherein the code is selected from a code protocol comprising ICD, CPT or HCPCS codes.

3. The method of claim 1 further comprising the steps of
(a) suggesting at least one code wherein a descriptor of the code correlates to the inputted patient medical condition; and
(b) at least one of the codes can be selected and assigned to the patient medical condition.

4. The method of claim 1 further comprising the steps of:
(a) entering the patient identifier and at least one code into the database;
(b) a search engine of the database initiating a search of the database for the patient identifier;
(c) upon identification of a matching patient identifier, the search engine initiating a search of the patient records within the database for the entered code.

5. The method of claim 4 wherein the search engine searches for codes within a family or class of codes wherein the family or class includes the assigned code.

6. The method of claim 5 wherein the assigned code is a specie of a larger group or genus of codes and the search engine searches the genus and species.

7. A database of patient identified medical information including medical codes assigned from past medical services where the included medical codes facilitate electronic searching and retrieval of patient medical information in real-time based upon current medical symptoms:
(a) a database of electronically searchable patient medical information wherein information is correlated by patient identifier;
(b) the information correlated to each patient identifier contains electronically searchable medical codes assigned on the basis of past health diagnoses, treatments or tests;
(c) means to electronically access the information correlated to a patient identifier contained in the database in real-time without human intervention including the steps of:
(i) inputting a patient identifier;
(ii) inputting descriptions of present patient medical conditions;
(iii) assigning medical codes correlated to the present patient medical conditions; and
(iv) searching the database utilizing the patient identifier and assigned codes to the present patient medical conditions.

8. The database of claim 7 further comprising electronically retrieving information from the database of past medical diagnoses, treatments or tests for the identified patient in real-time that pertains to the present patient medical conditions.

9. The database of claim 7 wherein the medical codes are derived from at least one medical code protocol comprising ICD, CPT or HCPCS code protocol.

10. The database of claim 7 wherein the database is accessible via a network or Internet.

11. The database of claim 7 comprises search functionality to include within the search codes having a parent-child, class-subclass or genus-species relationship to the assigned medical codes.

12. A database of patient identified medical information containing medical codes assigned from past medical services of the identified patient where the database includes a search functionality that allows a search request containing presently assigned diagnostic codes to include a search of past patient symptoms or diagnosis and to expand the search to include search of the identified patient medical information pertaining to past conducted treatment and test data relevant to the present symptoms and diagnosis of the identified patient comprising:
(a) an electronically searchable database comprising a non-transitory memory containing identified patient medical information wherein the patient medical information contains medical codes derived from a medical code protocol including ICD, CPT or HCPCS code protocols; and
(b) the electronically searchable database includes search functionality that allows a search request containing presently assigned diagnostic codes from the medical code protocol to electronically search and retrieve in real-time without human intervention patient identified medical information pertaining to past symptoms or diagnosis and relevant medical codes pertaining to past treatments or tests.

13. The database of claim 13 further comprising search functionality that includes artificial intelligence or machine learning.

14. The database of claim 13 wherein the search functionality comprises a correlated database.

15. A method of searching a database of patient's EHR information in real-time during the pendency of a patient encounter wherein the subject matter of the search is relevant or related to a present medical condition comprising:
(a) electronically entering a patient identifier and medical condition into a data field during a patient encounter;
(b) receiving one or more suggested codes of a medical code protocol correlated to the medical condition wherein the suggested codes are the product of steps of machine learning or artificial intelligence including:
(i) searching for a match of words within the medical condition and code descriptors correlated to the codes;
(ii) a correlated database utilizing the description of the medical condition that is correlated to medical terminology and correlated medical terminology is further correlated to relevant code descriptors;
(c) initiating a search of one or more EHR databases of patient identifiers and associated patient diagnoses, treatment or tests wherein the search comprises the patient identifier and at least one code;
(d) communicating the search to one or more EHR databases via a network or an Internet.

16. The method of claim 15 wherein a search result is communicated during the pendency of the patient encounter.

17. The method of claim 15 wherein the search of is conducted without human intervention.

18. The method of claim 15 wherein at least one code contained in the search is supplemented with at least one code of a differing code protocol using a search functionality of the database.

19. The method of claim 15 wherein the code searching includes utilizing a code cross-walking functionality of a database search functionality.
